(19) United States
(12) Patent Application Publication
(10) Pub. No.: US 2019/0205369 A1
(43) Pub. Date: Jul. 4, 2019

(54) METHOD AND SYSTEM OF DYNAMIC FORM GENERATION ON MOBILE DEVICES

(71) Applicant: Michael Andrew Iseri, Tustin, CA (US)

(72) Inventor: Michael Andrew Iseri, Tustin, CA (US)

(21) Appl. No.: 15/858,778

(22) Filed: Dec. 29, 2017

(51) Int. Cl. G06F 17/24; G06F 3/0482; G06F 3/0484

(52) U.S. Cl. CPC G06F 17/243; G06F 17/248; G16H 15/00; G06F 3/0484; G06F 3/0482

(57) ABSTRACT

A computer-based method in a mobile or stationary application, that dynamically generates an electronic form or file based on a user's responses that embodies at least one element that is dynamically generated that is not construed by any image templates or forms (i.e. customized instructions page for the user, creating unique power of attorney instructions, individualized consent forms, unique closing letters, etc.). The user provides at least one response through an application, such as through radio buttons and checkboxes, that is then utilized with other responses, to dynamically generate at least one element that is not based on template images or forms to create a unique final product or file containing at least that dynamically generated element. The computer-based method is designed to perform and function locally on the device without needing any servers, interne, cellular connection, or other network means to operate.

### COPYRIGHT

A portion of the disclosure of this patent document contains material that is subject to copyright protection. The copyright owner has no objection to the facsimile reproduction by anyone of the patent disclosure, as it appears in the Patent and Trademark Office patent files or records, but otherwise reserves all copyright rights whatsoever.

### TECHNICAL FIELD

The present invention relates generally to automatic document generation, and more particularly, to a system for generating a document using dynamic fields.

### BACKGROUND

Legal documents are needed in almost all areas of life. Most conventional legal procedures use template images or forms that have predefined fill-in-the-blank areas for a client or user to fill in. This process usually leads to an inefficient process that produces documents that contain unneeded and discarded legal language that could lead to other issues such as unnecessary legal rights or potential fraud. Most recently, software programs are being deployed that use predefined fields on a template image or form to allow users to input character strings into one or a series of fields. However, these predefined templates are limited since text cannot be added or removed from the template image or form other than the predesignated fields. These templates do not fully provide individuals seeking legal documents the ability to fully customize their legal needs. Further, unused fields on a template image or form can be later altered by anyone, thus leading to unrecorded alterations and fraud for these legal procedures.

Additionally, current forms rely on networked applications, which produce legal documents that are prepared from the forms vulnerable to network security breaches, thus jeopardizing the privacy of the user. Thus, there is need for a method for preparing specialized forms without relying on networks, servers, or any other apparatus besides the local device that contains the application to provide additional security and reliability. Since no networks such as the internet are needed by a localized application, all information can be controlled locally throughout the generation procedure, ensuring higher control for attorney-client confidentiality and informational security for all types of information being used for this legal procedure. This also would provide increased reliability to operate since network connectivity is not needed for such an application to operate.

Therefore, a computer-based method that that can dynamically create elements based on a user or client's responses is needed. A method that allows creating and removing significant portions on a legal document, without needing any prior template images or forms before finalizing the final product or file is needed to dramatically improve and optimize legal services for the future. After the final product or file is created, the user or client may do anything with the product or file such as printing or emailing.

### SUMMARY

According to one example, an apparatus for generating a document is disclosed. The apparatus includes a user input device, a display and a processor coupled to the user input and display. The processor is operable to execute a client application that is operative to display an interface on the display to request a response from a user. The application is operative to generate a dynamic element using the received response. The application is operative to insert the dynamic element in a field between static text for a document. The application is operative to generate a final document that includes the dynamic element. The application is operative to display the final document on the display.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is a block diagram of a system that may be used to generate a document using dynamic fields;

FIG. 2 is a block diagram of an example mobile user device for a person that may participate in the collection of emotional data by the system in FIG. 1;

FIG. 3 is a diagram representation of what a finalized product or file will generally contain;

FIGS. 4A-4F are screen images of the interface generated by the application in an example generation of an advance health care directive;

FIG. 5 is a screen image of a generated advance health care directive document without using dynamic data;

FIG. 6 is a screen image of a generated advance health care directive document with dynamic data;

FIGS. 7A-7F are screen images of various interfaces and the resulting document using a contract module incorporating dynamic data; and

FIG. 8 is a flow diagram of the process used by the dynamic element application operating on the user device in FIG. 1.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

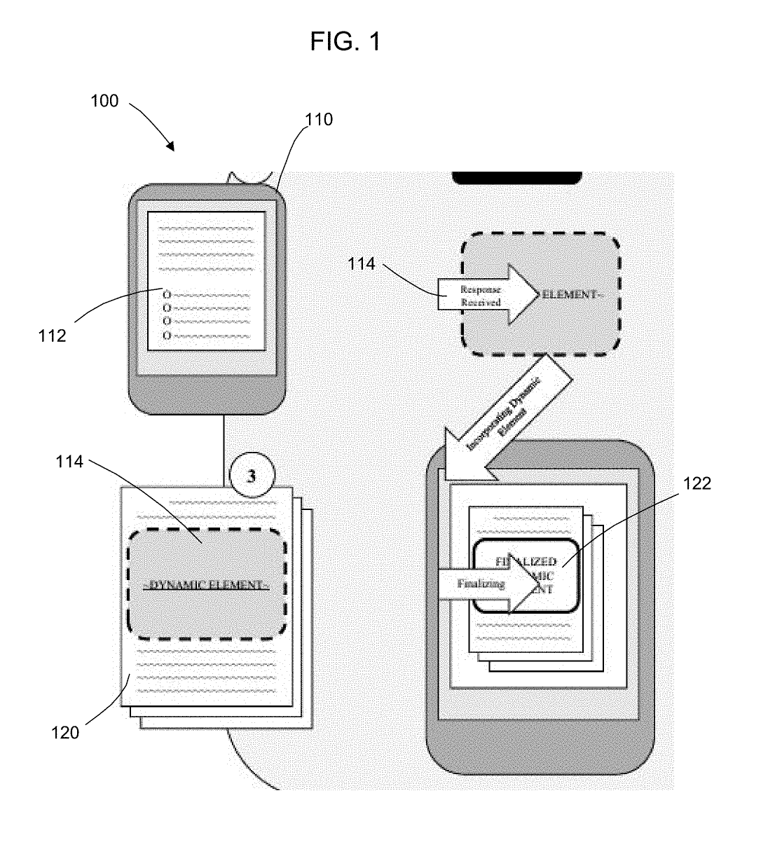

FIG. 1 shows four steps for the computer-based method to generate and incorporate at least one dynamic element to be used in a final product or file. A mobile device 110 runs an application that requests a response from a client or user. Based on the response (114), the application dynamically generates an element electronically or digitally. The generated dynamic element 114 is then incorporated into a product or file 120. The finalized dynamic element (122) may be used in a product or file to be used for other purposes by the user or client.

The application dynamically generates an electronic form or file based on a user's responses that embodies at least one element that is dynamically generated that is not construed by any image templates or forms. Examples of such a final document may include a customized instructions page for the user, unique power of attorney instructions, individualized consent forms, and unique closing letters. The user provides at least one response through an application, such as through radio buttons, word fields, buttons, checkboxes and other types of inputs. The response is then utilized with other responses, such as a client's name, to dynamically generate at least one element that is not based on template images or forms to create a unique final product or file containing at least that dynamically generated element. The computer-based method is designed to perform and function locally on the device without needing any servers, internet, cellular connection, or other network means to operate.

The mobile device 110 may include virtually any preferably mobile computing device may include portable devices such as cellular telephones, smart phones, display pagers, radio frequency (RF) devices, infrared (IR) devices, global positioning devices (GPS), Personal Digital Assistants (PDAs), handheld computers, wearable computers, tablet computers, integrated devices combining one or more of the preceding devices, laptops, and the like. As such, user devices running the application described below may range widely in terms of capabilities and features.

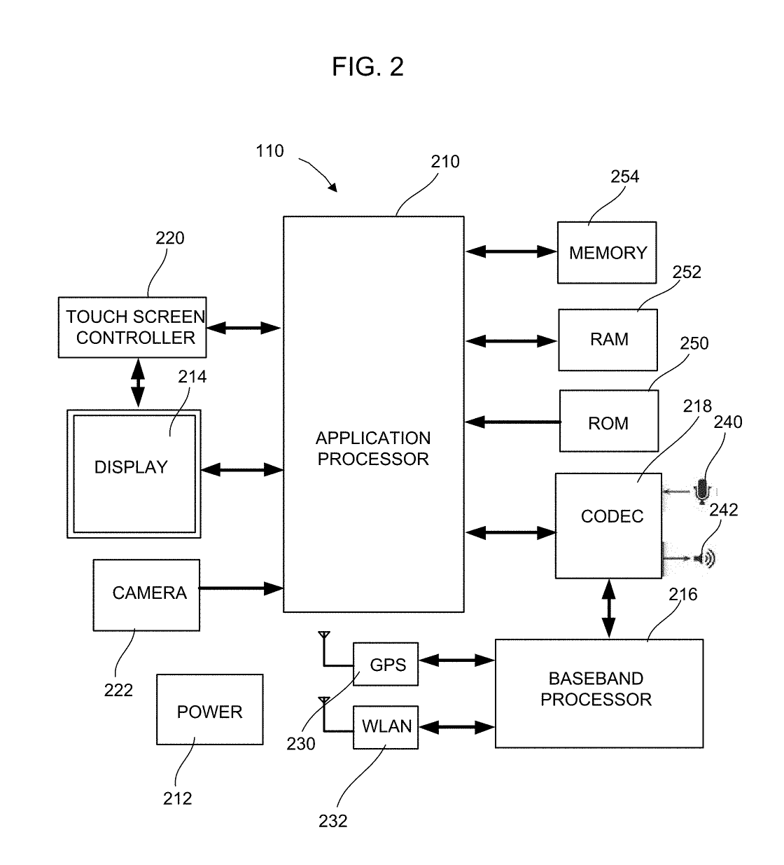

FIG. 2 is a block diagram of the components of a mobile user device such as the mobile user device 110 in FIG. 1. The mobile user device 110 includes an application processor 210, a power source 212, a display 214, a baseband processor 216, and a CODEC 218. In this example, the display 214 is an LCD touch screen that allows the user to control the applications run by the application processor 210 via touch inputs as well as view graphics generated by the application processor 210. The display 214 is controlled by a touch screen controller 220. The application processor 210 may be coupled to various devices such as a camera 222 and other interfaces such as a communication port, etc.

The baseband processor 216 receives signals from a network transmitter receiver 230 allowing communications with the network 108 in FIG. 1 and a geo-referencing receiver 232 that allows the reception of positioning data to determine the location of the mobile device 110. The baseband processor 216 processes in the signals and is coupled to the CODEC 218, which converts the signals for use by the application processor 210. The CODEC 218 also decodes audio signals received by a microphone 240 and encodes data signals for output by a speaker 242 for functions such as a telephone application run by the applications processor 210. Of course other audio devices such as a headset may be coupled through the CODEC 218.

The processors 210 and 216 may be conveniently implemented using one or more general purpose computer systems, microprocessors, digital signal processors, micro-controllers, application specific integrated circuits (ASIC), programmable logic devices (PLD), field programmable logic devices (FPLD), field programmable gate arrays (FPGA), and the like, programmed according to the teachings as described and illustrated herein, as will be appreciated by those skilled in the computer, software, and networking arts.

The operating system software and other applications are stored on read only memory (ROM) 250, random access memory (RAM) 252 and a memory storage device 254 for access by the applications processor 210. In this example, the memory storage device 254 is flash memory, but other memory devices may be used. The applications stored on the memory storage device 254 include the dynamic data document generation application, which creates interface graphics on the display to create a document.

The memory storage device 254 includes a machine-readable medium on which is stored one or more sets of instructions (e.g., software) embodying any one or more of the methodologies or functions described herein. The instructions may also reside, completely or at least partially, within memory storage device 254, the ROM 250, the RAM 252, and/or within the processors 210 or 216 during execution thereof by the mobile device 110. The instructions may further be transmitted or received over a network such as the network 108 in FIG. 1A via the network transmitter receiver 230. While the machine-readable medium is shown in an example to be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any medium that is capable of storing, encoding, or carrying a set of instructions for execution by the machine and that cause the machine to perform any one or more of the methodologies of the various embodiments, or that is capable of storing, encoding, or carrying data structures utilized by or associated with such a set of instructions. The term “machine-readable medium” can accordingly be taken to include, but not be limited to, solid-state memories, optical media, and magnetic media.

A variety of different types of memory storage devices, such as a random access memory (RAM) or a read only memory (ROM) in the system or a floppy disk, hard disk, CD ROM, DVD ROM, flash, or other computer readable medium that is read from and/or written to by a magnetic, optical, or other reading and/or writing system that is coupled to the processor, may be used for the memory or memories in the user device 110.

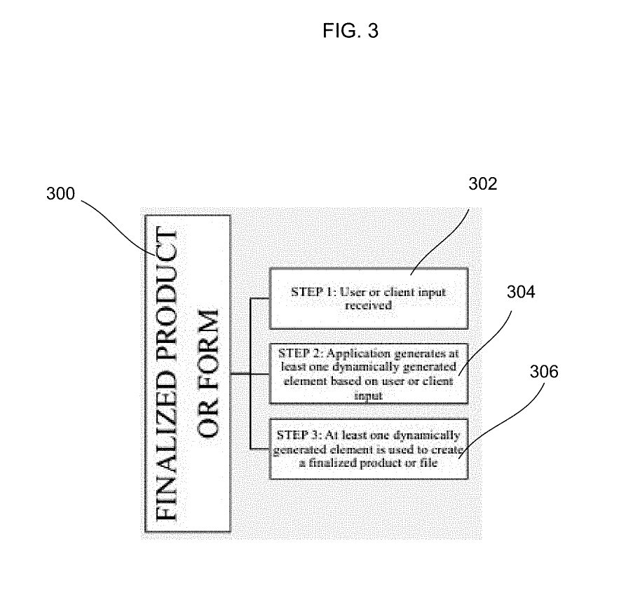

FIG. 3 is a block diagram that shows the components of a final product or file 300 generated through the computer-based method outlined in FIG. 1. The finalized product or file 300 is made by at least one user or client's response 302, a generated dynamic element based on the client or user response 304. The application then incorporates at least one dynamically generated element into a product or file 306. A product or file is produced and finalized that contains at least one dynamically generated element. In this example, the application is run using an Android operating system, but could be run on any other common operating systems and software languages such as iOS, Windows, Max OS, Linux, Java, Kotlin, C#, Swift, Basic, Pascal, COBOL, and so forth.

The process of creating the dynamic element in the overall application is as follows. The application is downloaded and installed with a template form or image. The application may be downloaded through a network, through an input port such as a USB port or copied from another storage media for example. The application can dynamically generate elements not based on template images or forms. The application requests and receives at least one response from a client or user to create at least one dynamically generated element not based on template images or forms. The application then incorporates this dynamic element into a product or file. The application then finalizes the product or file containing the dynamically generated element.

Thus, the application provides a process of creating dynamic elements through collecting information from a user through a segmented process involving multiple segments of one or more questions that adapts and changes based on a user's current and prior choices in order to collect more information from the user. An example of these adaptable segments of questions would be a user selecting the option under an advance healthcare directive to be kept alive if incapacitated in the hospital would then be presented with segmented questions on the methods of being kept alive and for how long. These questions are different if the user would have selected no. Once the module finished collecting information from a user, some or all information is presented to the user for verification and confirmation. Once the module finished collecting information from a user, then the responses are calculated and processed to create one or multiple dynamic elements to be added into the final product.

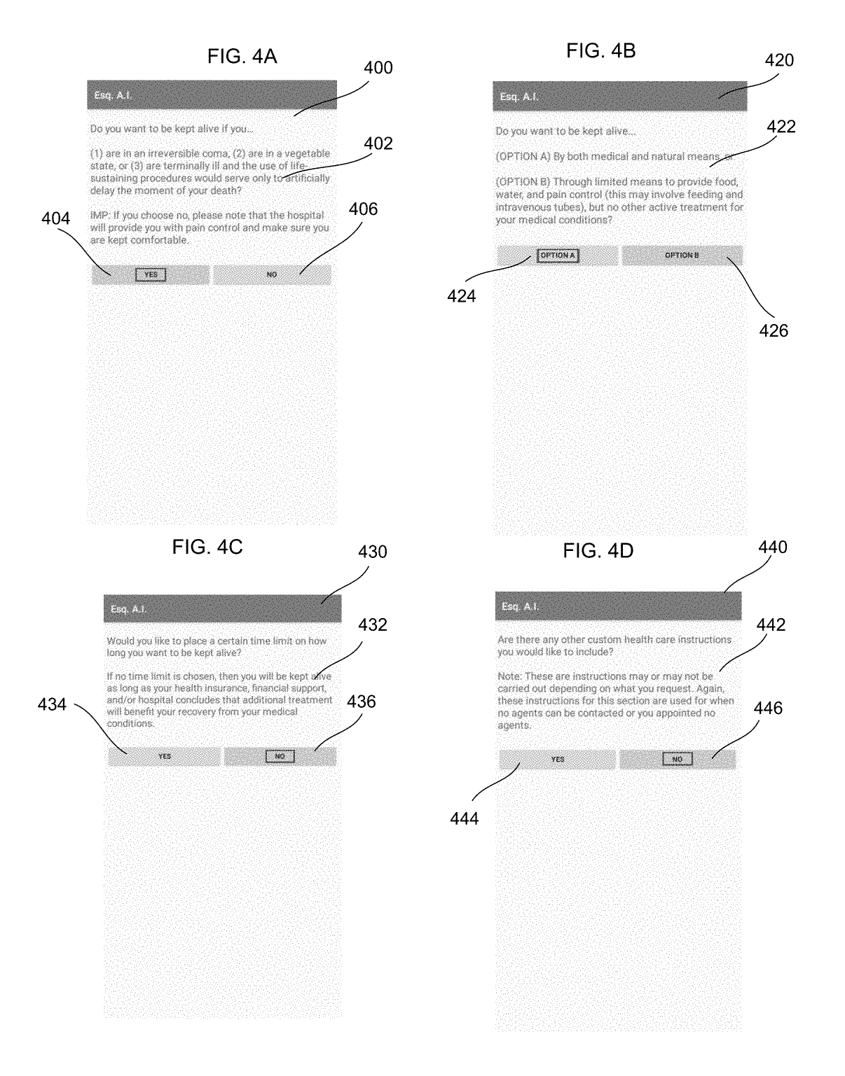

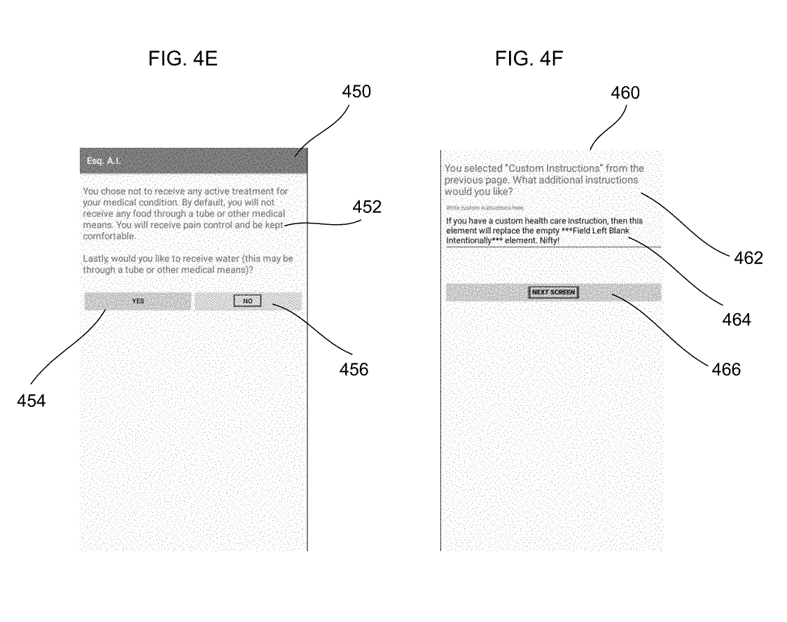

FIG. 4A is a screen image of an interface 400 generated by the application on a mobile device such as the mobile device 100 in FIG. 1 for generating an advance health care directive for health care. The screen image 400 includes informational text 402. In this example, the informational text 402 relates to legal options for an advance health care directive. The interface 400 includes a yes button 404 and a no button 406 that allows a user to select one of the legal options outlined in the informational text 402.

FIG. 4B is a screen image showing an interface 420 displayed by the application if the user selects the yes button 404. The yes button 404 initiates a request for an entry from the user as explained above. The interface 420 shows explanatory text 422 that explains two options for options to be kept alive in this example. The user may select between an option A button 424 and an option B button 426 that determines the conditions for being kept alive. Each of the options has an associated text that is incorporated in the final document.

FIG. 4C is a screen image showing an interface 430 displayed by the application after the user selects the options in the interface 420. The interface 430 includes explanatory text 432 that explains that the user may specify the amount of time the user wishes to be kept alive. The interface 430 includes a yes button 434 and a no button 436. If the user selects the yes button 434, a field will be displayed for a user to enter a date, for which time the user wishes to be kept alive. Once inputted, a dynamic element containing a set of customized instructions can then be generated based on the previous responses and inputs. The dynamic data in the form of the time to be kept alive is incorporated in the text of the final document.

FIG. 4D is a screen image showing an interface 440 displayed by the application after the user selects the options in the interface 430. The interface 440 includes explanatory text 442 that explains that the user may specify special instructions. The interface 440 includes a yes button 444 and a no button 446. If the user selects the yes button 444, a dynamic field will be displayed for a user to enter dynamic data. The entered dynamic data is incorporated in the final product after the user finishes the remaining interface screens described below.

FIG. 4E is a screen image showing an interface 450 displayed by the application if the user selects the no button 406 in the interface 400 in FIG. 4A. The interface 450 shows explanatory text 452 that explains the process of refusing food. The explanatory text 452 also includes instructions for options on whether to receive water. The user may select between a yes button 454 and a no button 456 for receiving water. Each of the options has an associated text that is incorporated in the final document after the user finishes the remaining interface screens described below.

FIG. 4F is a screen image showing an interface 460 displayed by the application after the user selects the options in the interface 440 to specify special instructions. The interface 460 includes an instructional text section 462 that instructs the user to enter custom instructions. The user may enter the custom instructions in a custom dynamic data field 464. The entered instructions are dynamic data that are included in the final document. The interface 460 includes a next screen button 466 that the user selects when the dynamic data is entered.

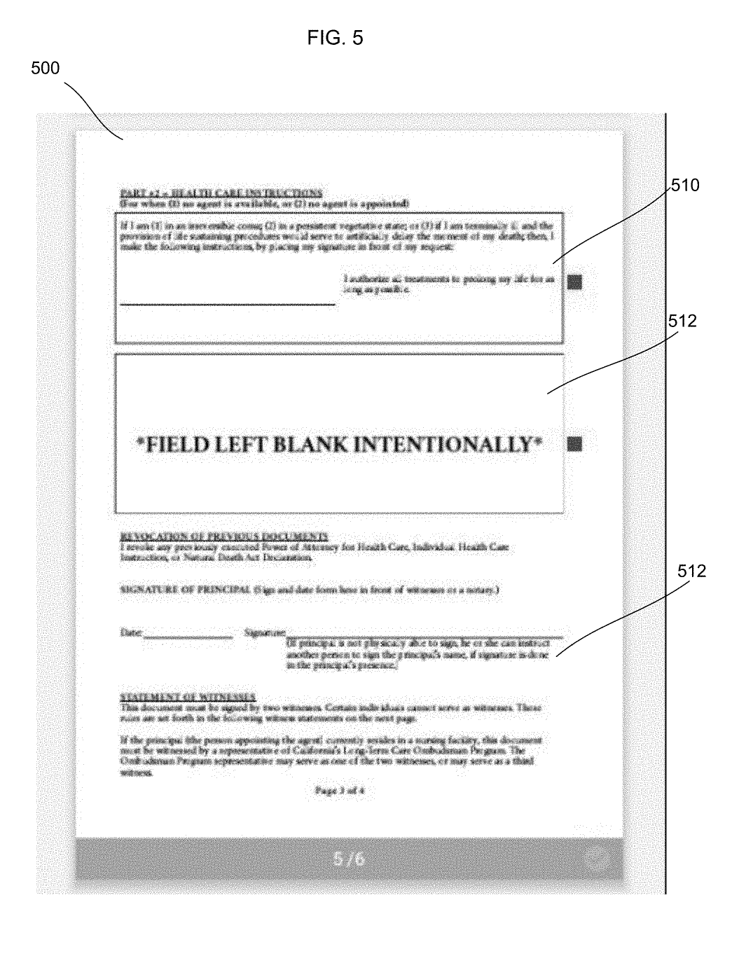

FIG. 5 is a screen image showing a final document 500 generated from information entered by the user in the interfaces shown in FIGS. 4A-4D. The final document 500 includes a static introduction text section 510 and another text section 512. Since the user selected the no button 446 in the interface 440 in FIG. 4D, there is no entered dynamic data in a dynamic data area 514.

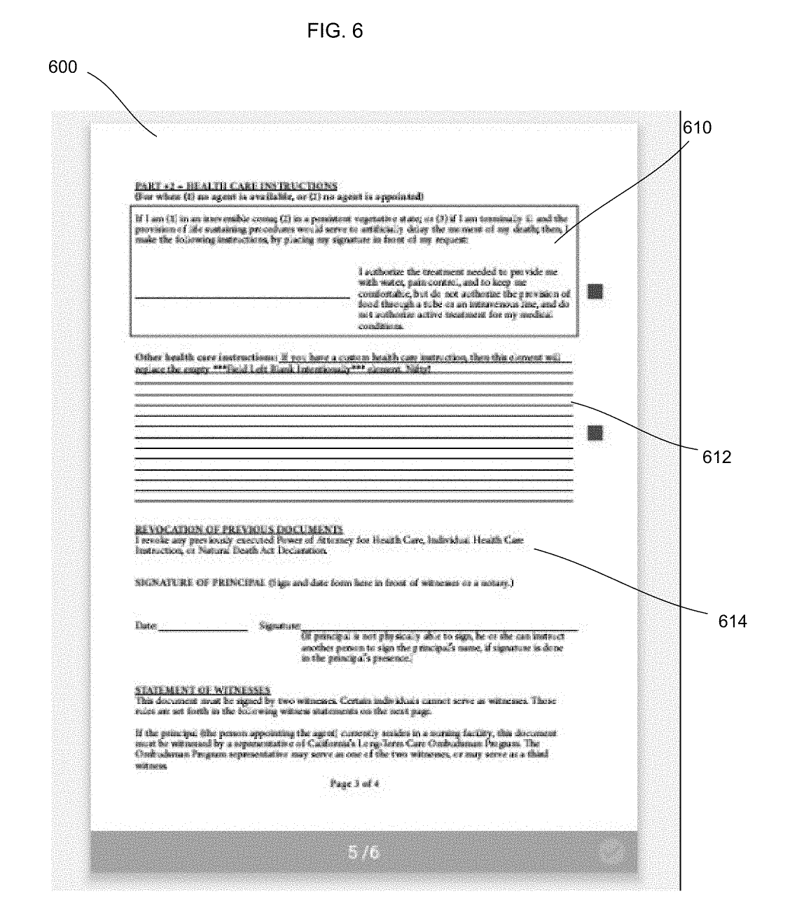

FIG. 6 is a screen image showing a final document 600 with two fixed text sections 610 and 614 and a dynamic data area 612. The dynamic data entered by the user in the interface 460 in FIG. 4F is displayed in the area 612.

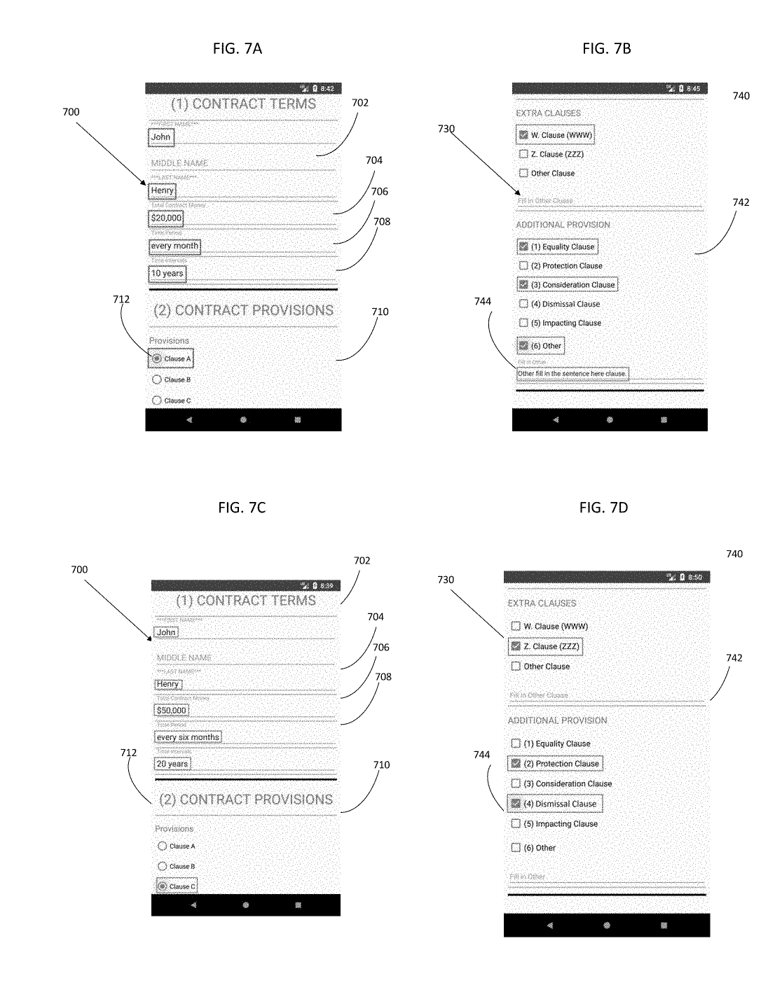

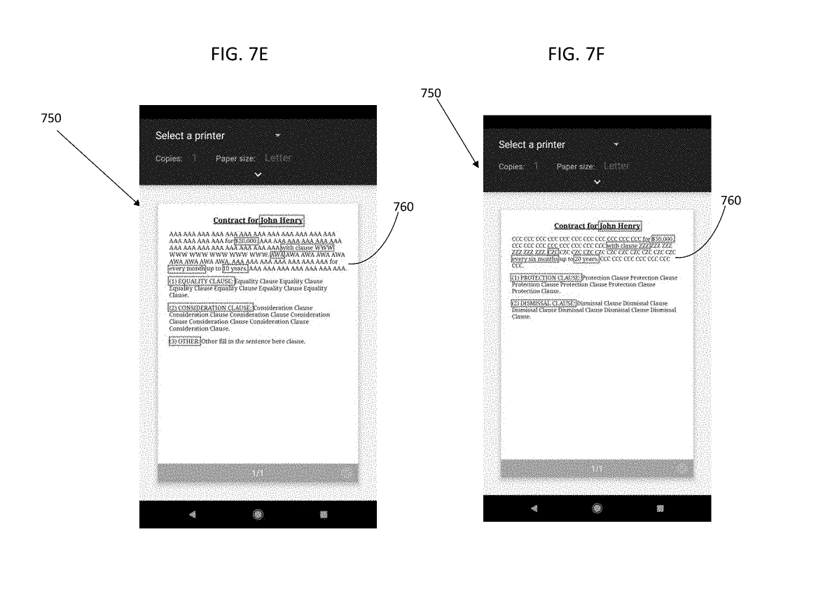

FIGS. 7A-7E show screen shots of the interfaces for another example of a dynamic form application that relates to allowing a user to draft a contract. The examples shown in FIGS. 7A-7E relate to a contract module that simplifies and highlights the focus of the dynamic elements process. The contract module may be executed with the application described above in relation to the mobile device 110 in FIG. 1. FIGS. 7A-7B shows interface screens that result in the production of one type of contract shown in FIG. 7E. FIGS. 7C-7D show the interface screens that result in the production of a different type of contract shown in FIG. 7F.

FIGS. 7A-B are screen images of the interfaces used to create a contract document. FIG. 7A is a screen image that shows an interface 700 for a user to enter relevant contract data. The interface 700 includes a name entry field 702, an amount entry field 704, a time period entry field 706, and a term period entry field 708. The interface 700 also includes a contract provisions field 710 that includes selection radio buttons 712 for different clauses that may be inserted in the contract. The clauses for each of the buttons 712 are designed to be commonly chosen clauses for the particular type of contract. Thus, each clause is predetermined at the least based on multiple permutations through user's choices with each clause being able to be further customized with user inputted text and objects (such as names and numerical figures) to further customize the clauses.

In this example, a user entering text for the first and last name being John Henry in the name entry field 702, $20,000 in the amount entry field 704, every month in the time period entry field 706, and 10 years in the time period entry field 708. The user has also selected the radio button corresponding to Clause A.

FIG. 7B is a screen shot showing a clause selection interface 730. The clause selection interface has an extra clauses field 740 and an additional provisions field 742. In this example, the extra clauses field allows the user to create dynamic elements that are inserted in the final document. The extra clauses checkboxes (W, Z, and Other) in the extra clauses field 740 are choices to further customize the previous provisions radio buttons 712 (Clause A, Clause B, and Clause C) to create a more complicated dynamic element. In this example, different checkboxes may include different W and Z clauses. The user may also select a fill in other clauses box in the extra clauses field 740 that when selected allows a user to enter text for additional clauses. The additional provisions field 742 includes pre-written provisions and corresponding checkboxes that a user may select to insert the pre-written provisions in the contract. For example, the additional provisions include an equity clause, a protection clause, a consideration clause, a dismissal provision, an impacting provision and an other box. A fill in text field 744 is included to allow a user to type in text for other provisions to be inserted when the other box is selected.

In this example, a user has selected the checkbox for the Clause (WWW) in the extra clauses field 740. The user has selected check boxes for the Equality Clause, the Consideration Clause, and the other box. Thus, the user may enter a sentence in the fill in text field 744 for the other provision.

FIGS. 7C-7D shows the resulting entries to generate a different contract using the same interfaces shown in FIGS. 7A-7B. In this example, a user has entered John Henry for the name entry field 702, $50,000 for the amount entry field 704, every 6 months for the period time entry field 706, and 20 years for the time period entry field 708. The user has selected Clause C in the radio buttons 712. In this example, the user has selected the checkbox for Clause (ZZZ) in the clause selection field 740. The user has selected different provisions such as a Protection Clause and a Dismissal Clause in the provisions selection field 742.

FIG. 7E is a screen image of an end product interface 750 displayed after the user submits the information from the interfaces shown in FIGS. 7A-7B. The application utilizes the information to create a dynamic and custom contract document with elements being added or removed. The interface 750 in FIG. 7E thus shows a finished document 760 that includes Clause A from the selection of the radio button from the radio buttons 712 in FIG. 7A, and a (WWW) clause selected from the extra clause field 740 in FIG. 7B. This creates a dynamic element in the contract as “AWA” and is inserted into the first paragraph of the document 760. The finished document 760 also includes the provisions selected from the additional provisions field 742 in FIG. 7B.

FIG. 7F is a screen image of the end product interface 750 reflecting a different contract created after the user submits the information from the interfaces shown in FIGS. 7C-7D. The generated document shown in the interface 750 in FIG. 7F is created after the user submits the information and utilizes the information to create a dynamic and custom document with elements being added or removed. The provisions selected in the provision field 742 in FIG. 7D are placed at the bottom of the contract in the interface 750 in FIG. 7F. Most notably, the radio button choice of Clause C from the buttons 712 in FIG. 7C and the checkbox Clause (ZZZ) selected in the extra clause field 740 in FIG. 7D create a dynamic element in the contract as “CZC” and is inserted into the first paragraph of the contract shown in the interface 750 in FIG. 7F.

The application contains a language module that allows a user to select instructions that are broadcast over the speaker 242 in FIG. 2 to assist the user in selections such as those shown in the interfaces shown in FIGS. 4A-4F. In this example, English is the default language used by the language module for oral instructions. A user may display an interface on the mobile device and select a different language. The language module will then broadcast the instructions for each interface in the selected language.

The present application holds all of the dynamic and static entries by the user as well as the final document on the mobile device. Thus, the present application avoids using a network such as the “cloud” to reference data used by the application. The document formation is entirely performed by the mobile device without needing any network resources. The processing and document formation is solely on the device itself.

Dynamic elements can be implemented and improve creation and customization for forms, pleadings, motions, procedures, instructions, addendum, memorandum, letters, and so forth for work and services in legal, medical, socials services, family affairs, veteran affairs, homeless affairs, governmental entities, non-governmental entities, nonprofits, and so forth. For example, dynamic elements in legal services could include, but not limited to, criminal matters, administration and agency procedures, civil matters, small claims matters, family law and dissolutions of marriage, domestic violence, elder abuse, veterans services, homeless services, medical and health related matters, estate planning, wills/trusts, power of attorney, business formation and incorporation, business legal affairs, bankruptcy, collections, trademarks, patents, and so forth. An application of dynamic elements for a power of attorney for healthcare, for example, could use information collected from the user throughout a module to create customized, dynamic elements to be used for individualized instructions on how to be treated in a hospital due to being brain dead and customized instructions and closing letter on how to proceed with that and other options.

The produced documents from the compilation of dynamic elements may be output in any format. Dynamic elements cover, but are not limited to, .pdf (Portable Document File), .doc (document file format) and .docx (xml document file format), .odt (OpenDocument Text document file), .rtf (Rich Text Format), .txt (text file), .wpd (WordPerfect document), .jpg/.jpeg and variants (Joint Photographic Experts Group), .bmp (bitmap), .tif or .tiff (Tagged Image File Format), .raw (raw image format), .png (Portable Network Graphics), .gif (Graphics Interchange Format), and so forth.

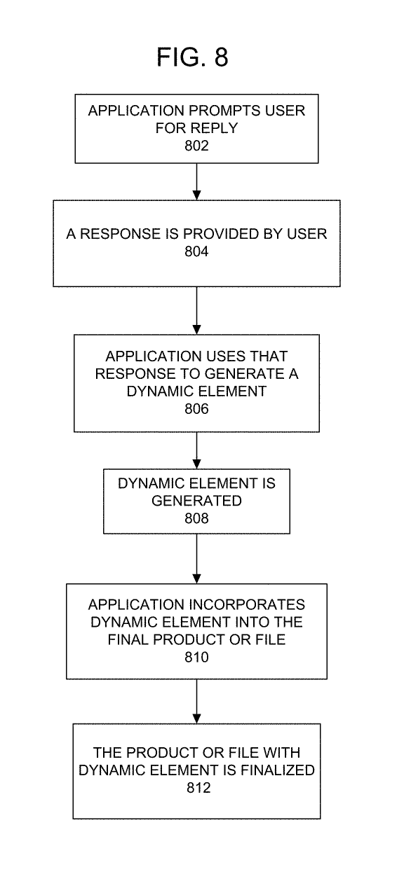

FIG. 8 shows a flow diagram of the code executed by mobile device 110 in FIG. 2 to generate a document based on dynamic elements provided by the user. The flow diagram in FIG. 9 is representative of example machine readable instructions for the mobile device. In this example, the machine readable instructions comprise an algorithm for execution by: (a) a processor, (b) a controller, and/or (c) one or more other suitable processing device(s). The algorithm may be embodied in software stored on tangible media such as, for example, a flash memory, a CD-ROM, a floppy disk, a hard drive, a digital video (versatile) disk (DVD), or other memory devices, but persons of ordinary skill in the art will readily appreciate that the entire algorithm and/or parts thereof could alternatively be executed by a device other than a processor and/or embodied in firmware or dedicated hardware in a well-known manner (e.g., it may be implemented by an application specific integrated circuit (ASIC), a programmable logic device (PLD), a field programmable logic device (FPLD), a field programmable gate array (FPGA), discrete logic, etc.). For example, any or all of the components of the interfaces could be implemented by software, hardware, and/or firmware. Also, some or all of the machine readable instructions represented by the flowchart of FIG. 8 may be implemented manually. Further, although the example algorithm is described with reference to the flowcharts illustrated in FIG. 8, persons of ordinary skill in the art will readily appreciate that many other methods of implementing the example machine readable instructions may alternatively be used. For example, the order of execution of the blocks may be changed, and/or some of the blocks described may be changed, eliminated, or combined.

FIG. 8 shows a flow diagram of a general step-by-step procedure of how a user or client generates a dynamic element through the application running on the mobile device 110. The application first prompts the user to provide at least one response that is used to generate a dynamic element. (802). The user or client provides at least one response via the input devices for the mobile device 110. (804). At least one of the user or client's response is used by the application to generate a dynamic element not based on any template images or forms (806). The dynamic element not based on any template images or forms is generated (808). The application then incorporates the generated dynamic element into a product or file such as a legal document (810). The product or file is finalized containing at least one dynamic element from this computer-based method (812).

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

Computing devices typically include a variety of media, which can include computer-readable storage media and/or communications media, in which these two terms are used herein differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

1. An apparatus for generating a document comprising:
a user input device;
a display; and
a processor coupled to the user input and display, the processor operable to execute a client application, the client application operative to:
display an interface on the display to request a response from a user;
generate a dynamic element using the received response;
insert the dynamic element in a field between static text for a document;
generate a final document that includes the dynamic element; and
display the final document on the display, wherein the generation of the dynamic element and final document are performed exclusively by the apparatus without an external communication from a network.

2. The apparatus of claim 1, wherein the document is in a portable document format.

3. The apparatus of claim 1, wherein the client application is installed and stored locally on the apparatus and not on an external network.

4. The apparatus of claim 1, wherein the client application operates exclusively on the apparatus.

5. The apparatus of claim 1, wherein the dynamically generated element is at least one-character string or object that was generated from a user input from the user input device.

6. The apparatus of claim 1, wherein the client application includes an interface having a radio button, a checkbox, word field, or a button to generate another element for the document.

7. The apparatus of claim 1, wherein the document is an advanced health care directive.
